SEQUENCE LISTING (3 sequences)

SEQ ID NO: 1 - protein, 23 aa, Artificial Sequence
  Synthetic
IEEQAKTFLDKFNHEAEDLFYQS

SEQ ID NO: 2 - protein, 41 aa, Artificial Sequence
  Synthetic
SVTCKSGDFSCGGRVNRCIPQFWRCDGQVDCDNGSDEQGCP

SEQ ID NO: 3 - protein, 39 aa, Artificial Sequence
  Synthetic
PKTCSQDEFRCHDGKCISRQFVCDSDRDCLDGSDEASCP

(19) United States
(12) Patent Application Publication
(10) Pub. No.: US 2022/0041660 A1
(43) Pub. Date: Feb. 10, 2022

(54) COMPOSITIONS AND METHODS FOR SEQUESTERING VIRAL PARTICLES IN RESPIRATORY TRACT

(71) Applicants: Jason P. Gleghorn, Lincoln University, PA (US); Catherine A. Fromen, Wilmington, DE (US); Katherine M. Nelson, Elkton, MD (US)

(72) Inventors: Jason P. Gleghorn, Lincoln University, PA (US); Catherine A. Fromen, Wilmington, DE (US); Katherine M. Nelson, Elkton, MD (US)

(73) Assignee: University of Delaware, Newark, DE (US)

(21) Appl. No.: 17/396,006

(22) Filed: Aug. 6, 2021

Related U.S. Application Data
(60) Provisional application No. 63/061,862, filed on Aug. 6, 2020.

(51) Int. Cl. C07K 14/005

(52) U.S. Cl. CPC C07K 14/005; A61K 38/00

(57) ABSTRACT

A composition for sequestering a pathogen, for example, viral particles of a target virus in the respiratory tract of a subject is provided. The composition comprises an effective amount of sequestering particles having a protein binding agent on the surface of the sequestering particles. The protein binding agent binds a pathogenic surface protein, for example, capsid protein on the surface of the viral particles. The sequestering particles and the target pathogen, for example, viral particles, form aggregates. Also provided is the use of the composition for sequestering a target pathogen, for example, viral particles of a target virus, in the respiratory tract of a subject.

### STATEMENT REGARDING FEDERALLY SPONSORED RESEARCH

This invention was made with government support under Grant No. U54-GM104941 awarded by Delaware Clinical and Translational Research ACCEL Program funded by the National Institutes of Health. The government has certain rights in the invention.

This application claims priority to U.S. Provisional Patent Application No. 63/061,862, filed Aug. 6, 2020, the contents of which are incorporated herein by reference in their entireties for all purposes.

### REFERENCE TO SEQUENCE LISTING

The present application is being filed along with a Sequence Listing in electronic format. The Sequence Listing is provided as a file entitled UOD-525US_SequenceListing.txt, created Aug. 4, 2021, which is 1,532 bytes in size. The information in the electronic format of the Sequence Listing is incorporated herein by reference in its entirety.

### BACKGROUND OF THE INVENTION

The COVID-19 pandemic is estimated to cost Americans $16 trillion largely due to our inability to control the spread of the virus and treat patients, which extends the length of the pandemic. The novel respiratory coronavirus, severe acute respiratory syndrome coronavirus 2 (SARS-CoV-2), causes the disease COVID-19 and has been the subject of much interest and research in the recent months. The COVID-19 pandemic has made it evident that current standard medical intervention to protect at-risk populations and reduce the spread of respiratory viruses is severely limited. SARS-CoV-2 is just the latest example of such an outbreak, in which face coverings and physical distance are the only prophylactic measures at our disposal. SARS-CoV-2 is particularly infectious and up to 60% of infected people have mild symptoms or are asymptomatic, resulting in a high rate of transmission. This spread is further exacerbated by limited efficacy and availability of testing. These factors, both biological and societal, leave the populace in a vulnerable position as the virus can be transmitted person to person unknowingly with ease and efficiency. However, the COVID-19 pandemic is not an isolated situation but is just the most recent example of a respiratory pathogen causing global devastation, with other examples including the H1N1, SARS, and MERS pandemics, and significant global deaths from tuberculosis infections.

The COVID-19 pandemic continues to threaten lives around the world and remains a challenge to contain due to asymptomatic spread. The lack of preparation in terms of proactive medical intervention has been laid bare to see, and significant improvements need to be made to prepare for the next viral outbreak, which will certainly come. A prophylactic approach would decrease viral spread and infection severity of respiratory viruses. Unfortunately, this current pandemic has proven that non-pharmacological interventions are not always implemented or followed leaving many populations at risk and desperate for an alternative before a vaccine can be employed.

ACE2 binding enables SARS-CoV-2 cellular entry and is implicated in COVID-19 disease pathology. The novel respiratory coronavirus SARS-CoV-2 enters cells via spike (S) protein binding to the angiotensin converting enzyme (ACE) 2 protein. Interestingly, ACE2 is downregulated by SARS-CoV-1, and a similar mechanism is believed to occur in SARS-CoV-2 infection, contributing to lung injury. During host immune response, most patients develop neutralizing antibodies targeting the spike (S) protein, implicating the importance of blocking the ACE2 binding event in overcoming COVID19 infection. However, antibodies against SARS-CoV-2 are not observed in plasma until an average of 5 days (IgM, IgA) or 14 days (IgG) after symptoms begin. With symptom onset lagging from exposure by a median of 5.1 days and spanning as long as 11.5 days, neutralizing antibody development is delayed, leaving ample time for viral replication and asymptomatic transmission. There is a critical need to develop post-exposure, prophylactic measures that can intervene in this critical time period prior to antibody development.

While it is truly remarkable that a vaccine was able to be developed against SARS-CoV-2 within one year, this is not the case for all respiratory infections. Even with the rapid vaccine development, the virus has still been able to spread relatively unchecked for this past year. With yet more time required to vaccinate a sufficient percentage of the population to be effective, the virus continues to rage on. There is an urgent need for the development of a widely applicable prophylactic for respiratory viral infections, as well as bacterial and fungal infections, that can be given before or once contact with an infected individual is suspected to reduce the spread of the pathogen.

### SUMMARY OF THE INVENTION

The invention relates to novel compositions and methods for sequestering particles of a target pathogen in the respiratory tract of a subject. The invention is based on the inventors' surprising discovery of sequestering particles that recognize and form aggregates with viral particles.

A composition for sequestering viral particles of a target virus in the respiratory tract of a subject is provided. The composition comprises an effective amount of sequestering particles having a protein binding agent on the surface of the sequestering particles. The protein binding agent binds a capsid protein on the surface of the viral particles. The sequestering particles and the viral particles form aggregates.

The sequestering particles may be selected from the group consisting of polymeric particles, liposomes, lipid/membrane-wrapped particles, alginate, polysaccharide-based platforms, and combinations thereof. The sequestering particles may comprise poly(ethylene glycol) diacrylate (PEGDA).

The target virus may be a respiratory virus selected from the group consisting severe acute respiratory syndrome coronavirus 2 (SARS-CoV-2), influenza virus, respiratory syncytial virus (RSV), hantavirus, rhinovirus, parainfluenza virus (PIV), and human metapneumonia virus (hMPV).

The target virus may be SARS-CoV-2, the capsid protein may be a spike (S) protein and binds a S protein binding domain in angiotensin converting enzyme (ACE) 2 protein, and the protein binding agent may be a peptide consisting of an amino acid sequence at least 80% identical to the S protein binding domain and blocks the binding of the S protein to the ACE2 protein. The peptide may consist of the amino acid sequence of C-(PEG)₄-IEEQAKTFLDKFNHEAEDLFYQS (SEQ ID NO: 1), C-SVTCKSGDFSCGGRVNRCIPQFWRCDGQVDCDNGSDEQGCP (SEQ ID NO: 2), or C-PKTCSQDEFRCHDGKCISRQFVCDSDRDCLDGSDEASCP (SEQ ID NO: 3).

The composition may comprise the protein binding agent at 10-50 wt %, based on the total weight of the composition. The sequestering particles may have a particle size of 1-10 μm. The sequestering particles may have a particle size of 1-5 μm. The sequestering particles may have a particle size of 5-10 μm.

The sequestering particles may be desiccated. The sequestering particles may have a zeta potential less than zero. The sequestering particles may have a surface modification selected from the group consisting of PEGylation, zwitterions, and CD47. The composition may be in a mixture of a hydrogel and dry powder.

The composition may be formulated for aerosolized or nebulization delivery. The composition may be formulated for oral and/or nasal administration. The subject may have been vaccinated against the target virus.

A method for sequestering viral particles of a target virus in the respiratory tract of a subject is also provided. The method comprises delivering the composition of the present invention to the respiratory tract of the subject; and forming aggregates of the sequestering particles and the viral particles in the respiratory tract. The viral particles are sequestered by the sequestering particles. The method may further comprise removing the aggregates from the subject. The method may further comprise reducing at least 50% of the viral particles in the respiratory tract of the subject.

### BRIEF DESCRIPTION OF THE DRAWINGS

FIG. 1 shows that microparticles (MPs) comprised of PEGDA block viral binding in an analogous manner to neutralizing antibodies.

FIG. 2 shows that MPs decorated with a binding peptide can be delivered via inhalation.

FIG. 3 shows schematic of the chemistry to cross-link the particles in A. FIG. 3B shows an SEM of the particles showing that they can be produced and are uniform. FIG. 3C shows that increasing amount of AEM in the composition will increase the amount of peptide (ACE2) that is able to be covalently bound to the particle. FIG. 3D shows the zeta-potential of the particles are all negative. FIG. 3E shows that a fluorescent tag can be incorporated into the formulation and used to image the particles.

FIG. 4 shows that the particles can bind to virus based on presence of SYBR on the particles using flow cytometry. SYBR stains for DNA in virus. As concentration of ACE2 peptide, more particles fluoresce green from SYBR indicating that virus has bound to the particles as a function of peptide concentration-not just non-specific binding.

FIG. 5 shows that the ability for the particle to sequester the virus is specific. FIG. 5A shows that a control virus which has the ability to infect cells but does not express spike on its surface infects cells after being incubated with particles. FIG. 5B shows that virus that can infect cells but does express spike on its surface infects cells at a much lower frequency than the control virus after incubation with the particles.

### DETAILED DESCRIPTION OF THE INVENTION

The present invention provides compositions and methods for sequestering pathogens, for example, viral particles of target viruses, bacteria or fungi, in the respiratory tract of a subject before or upon contact with the target pathogens (e.g., viruses). This invention is based on the discovery by the inventors of sequestering particles decorated with protein binding agents on the surface of the sequestering particles capable of sequestering viral particles such that the sequestered viral particles may be removed from the body of the subject. This prophylactic measure would help to reduce the viral spread which would allow more time for vaccine development. The vaccine is also most effective when given to a population without widespread infection, so an additional measure to reduce viral spread could help make the vaccine even more effective once brought to the public. The inventors have created a platform technology to be able to sequester viruses from the upper airway using modular particles that can be easily tuned to bind to any virus. Such a system could be rapidly modified upon identification of a new virus, on a time scale that is faster than that to create a novel functional vaccine. Such a prophylactic could protect at risk populations by sequestering virus in those who have potential exposure, thus reducing transmission. By limiting uncontrolled spread, this provides more time for vaccine development. Of additional importance, viral load is well correlated to disease outcome in many infections, including COVID-19. This proposed prophylactic would function to sequester viral load in the airway which would not only limit transmission, but would reduce the degree of infection, thus likely reducing the duration and/or need for hospitalization and decreasing the burden on hospitals. Overall the prophylactic works in many ways to reduce the cost and impact of a respiratory pandemic, and by helping to save lives.

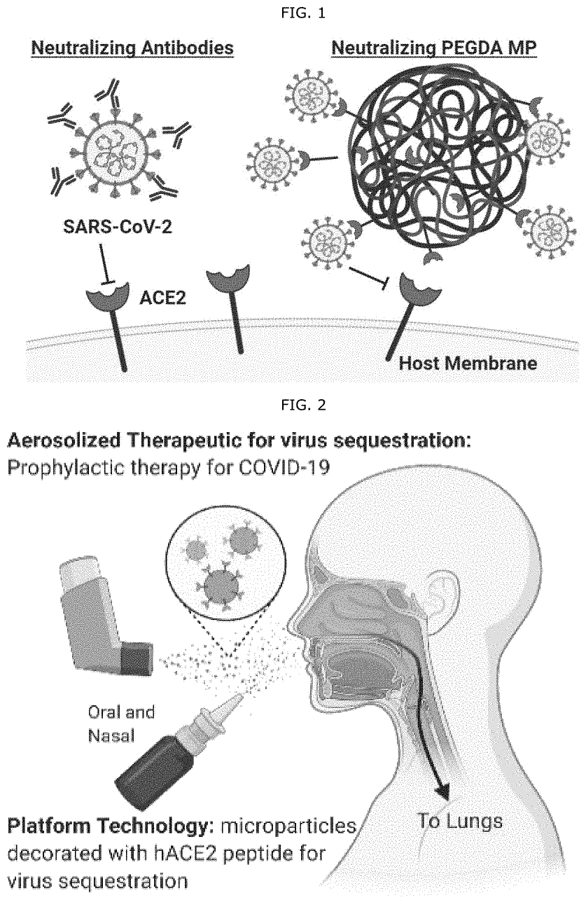

The newly discovered sequestering particles can be used to develop a treatment platform targeted against pulmonary infections by pathogens (e.g., viruses, bacteria or fungi). Microparticles (MPs) decorated with a peptide that is capable of binding and sequestering viral particles, bacteria or fungi may be synthesized to reduce infectivity. While delivery into the airspace of the lung is a more efficient and direct route to the location of infection, there are a variety of obstacles the platform faces to be effective, including correct sizing of particles to control the location of deposition, mucocilliary clearance mechanisms, and potential macrophage infection. The inventors have discovered that aerosolized delivery of 5-10 μm inert poly(ethylene glycol) diacrylate (PEGDA) MPs decorated with a peptide to bind pathogenic surface proteins (e.g., viral entry proteins) will sequester free pathogens (e.g., viruses) in the upper airway, decrease pathogenic load, and be safely cleared from the lung (FIGS. 1 and 2). The peptide decorating the surface of the MP can easily be swapped and modified to specifically bind to any target respiratory pathogens, including viruses, like SARS-CoV-2. This establishes a modular treatment platform for pathogenic pulmonary infections. This platform can be applied to treat any respiratory pathogenic infections, including respiratory viral infection, from other zoonotic crossover events to the seasonal flu.

The platform treatment involving the sequestering particles would allow those at high risk of exposure (healthcare professionals, teachers, essential workers), and high-risk populations (nursing homes, elderly, immunocompromised), to have a therapeutic option in the case of exposure to limit or potentially even prevent pathogenic infection such as viral infection, which may save lives. In this novel method, an inhalable MP platform system is created for prophylactic treatment following exposure that will sequester pathogens (e.g., viruses) directly at the site of infection and limit off-target effects. This is significant because the modularity of the treatment allows for modification to treat many different pathogenic infections, for example, viral infections. The platform would be able to be developed rapidly, as usually one of the first pieces of knowledge gained about a novel pathogen, for example, virus, bacterium or fungus, is its binding and cell entry mechanism, which can then be harnessed in this platform for viral sequestration. Furthermore, this robust and interchangeable design of a drug delivery system for sequestration of pathogens (e.g., viruses) is a platform technology; substitution of the specific pathogen binding peptides, for example, viral binding peptides, can be used as a rapid strategy against other pathogenic outbreaks. As observed with COVID-19, despite the relatively rapid vaccine development, there is a significant lag time with vaccine production, but the design of sequestering microparticles to a novel pathogen (e.g., virus) can be performed in a relatively short time span through both easy design and fast-tracking through pre-clinical trials. This treatment will be able to be disseminated to at risk populations therefore limiting the spread of a future pathogenic outbreak. The inventors' design has impacts on not only a treatment for COVID-19 but also as applied to other novel and existing pathogenic infections.

The prophylactic approach discovered by the inventors would inhibit inhaled pathogens (e.g., viruses) from entering pulmonary epithelial cells, as opposed to other mechanisms such as inhibiting RNA polymerase or endosomal escape, and lessen the pathogenic load (e.g., viral load) in the lung, not only helping the individual's disease prognosis, but decreasing their infection potential.

The inventors have engineered therapeutic microparticles capable of effectively navigating the complex lung physiology to sequester pathogens (e.g., viruses) and reduce pathogenic load (e.g., viral load). A peptide fragment of ACE2 corresponding to the binding region of SARS-CoV-2 has been shown to be stable and able to bind to the virus with good selectivity in vitro.

Further, the inventors have discovered that this approach with a desiccated particle and controlled regional delivery to the lung would limit negative immune response and microparticle rehydration would aid in edematous fluid clearance. Aerosols with larger aerodynamic diameters (10 μm) deposit in the upper respiratory tract (mouth, pharynx and trachea) and microparticles 1-5 μm in diameter deposit in the lower bronchi and alveoli regions of the lung. Objects, particularly deposited in the lower airways, are cleared from the lung by coughing, mucociliary transport, or phagocytes.

The invention provides a composition for sequestering a pathogen, for example, viral particles of a target virus, bacterium or fungus, in the respiratory tract of a subject. The composition comprises an effective amount of sequestering particles. The sequestering particles have a protein binding agent on the surface of the sequestering particles. The protein binding agent binds a pathogenic surface protein, for example, a capsid protein on the surface of the viral particles. As a result, the sequestering particles and the pathogen, for example, viral particles, form aggregates.

The term “pathogen” used herein refers to a microorganism capable of causing a disease or condition in a host. Examples of pathogens include viruses, bacteria and fungi.

The term “virus” used herein refers to a microorganism that is smaller than a bacterium and replicates only in living cells of a host. A virus exists in the form of particles, or virions, each composed of a viral genome, which may be RNA or DNA, encoding viral proteins, a protein coat, also known as capsid, enclosing the viral genome, and, in some cases, an outside envelope of lipids. A viral particle attaches to a host cell via interaction between the capsid protein and one or more specific receptors on the surface of host cell (“cellular receptors”). Upon attachment, the viral particle releases its viral genome into the host cell, and the host cell is infected. In the host cell, the viral genome is replicated, viral proteins are expressed, and new viral particles are assembled in the host cells before being released from the host cells.

The target pathogen may be a respiratory pathogen. The target pathogen may be a respiratory virus, bacterium or fungus. Examples of respiratory viruses include severe acute respiratory syndrome coronavirus 2 (SARS-CoV-2), influenza virus, respiratory syncytial virus (RSV), hantavirus, rhinovirus, parainfluenza virus (PIV), and human metapneumonia virus (hMPV). Examples of respiratory bacteria include Streptococcus pneumoniae, Haemophilus species, Staphylococcus aureus, Nontuberculous mycobacteria, and Mycobacterium tuberculosis. Examples of respiratory fungi include Aspergillus, Cryptococcus, and Pneumocystis.

The term “protein binding agent” used herein refers to an agent that is capable of binding a pathogenic surface protein, for example, capsid protein, or peptidoglycan and is not the full native cellular receptor on a host cell for the pathogenic surface protein. The protein binding agent may be selected from the group consisting of peptides, aptamers, antibodies, nanobodies, and a combination thereof.

A peptide is a polymer of 10-20 amino acids. The peptide may have an amino acid sequence at least 70, 75, 80, 85, 90, 95, 99 or 100% identical to that of a binding domain on a cellular receptor specific for a capsid protein of a viral particle. The peptide sequence may depend on the pathogenic surface protein, for example, capsid protein, and thus unique to each pathogen, for example, virus.

An aptamer is a short single-stranded synthetic oligonucleotide that demonstrates high binding affinity and specificity. The aptamer is usually from 20 to 60 nucleotides in size. The aptamer sequence may depend on the pathogenic surface protein, for example, capsid protein, and thus unique to each pathogenic surface protein, for example, virus.

An antibody is an immunoglobulin Y-shaped protein with two fragment antigen binding (Fab) regions that bind to specific antigens of a capsid protein of a viral particle. Nanobodies are heavy chain antibodies with a single variable domain that bind to specific antigens of a capsid protein of a viral particle. The Fab region on the antibody or nanobody may have an amino acid sequence at least 70, 75, 80, 85, 90, 95, 99 or 100% identical to that of a binding domain from monoclonal antibodies for a capsid protein of a viral particle. The antibody and nanobody sequence may depend on the pathogenic surface protein, for example, capsid protein, and thus unique to each pathogenic surface protein, for example, virus.

The term “sequestering” used herein refers to forming aggregates with a pathogen, for example, viral particles of a virus, and thus preventing the pathogen, for example, viral particles, from attaching and infecting host cells. The sequestering may be accomplished by introducing a protein binding agent capable of binding the pathogenic surface protein, for example, capsid protein on the surface of the viral particles, to preventing the pathogenic surface protein, for example, capsid protein, from binding specific receptors on the surface of host cells.

The term “sequestering particles” used herein refers to synthetic particles capable of sequestering a pathogen, for example, viral particles, by forming aggregates and thus preventing the pathogen, for example, viral particles, from attaching to host cells. The sequestering particles do not aggregate with each other. The sequestering particles allow for further surface functionalization with a protein binding agent. The sequestering particles are biocompatible, and are naturally degraded and/or cleared from the airspaces and/or body of a subject. The sequestering particles may swell in aqueous conditions.

The sequestering particles may be selected from the group consisting of polymeric particles, liposomes, lipid/membrane-wrapped particles, alginate, polysaccharide based platforms, and combinations thereof. The polymeric particles may comprise poly(ethylene glycol) diacrylate (PEGDA), poly(acrylic acid), poly lactic-co-glycolic acid, 2-carboxyethyl acrylate or 2-amino ethyl methacrylate. The liposomes are spherical droplets stabilized by a phospholipid membrane. The lipid/membrane-wrapped particles are synthetic particles such as gold, silver, silica, or PEGDA coated with cellular membranes harvested from mammalian cells. The polysaccharide based platforms are particles made from natural hydrogel materials such as starch, alginate, and agarose. The sequestering particles may be swellable biocompatible hydrogel, such as PEGDA, alginate, agarose, hyaluronic acid, gelatin, poly(acrylic acid). The sequestering particles may further comprise a dye or fluorescent molecule.

The sequestering particles may be microparticles, nanoparticles or a combination thereof. The term “particle size” used herein refers to an average diameter of a particle. The term “microparticles” used herein refers to particles having a particle size of at least 750 nm or 1 μm. The term “nanoparticles” used herein refers to particles having a particle size of less than 750 nm or 1 μm. The particle size may be used to control the location of deposition of the sequestering particles. The sequestering particles may have a particle size of 1-10 μm. The sequestering particles may have a particle size of 5-10 μm, which may be suitable for upper airway delivery. The sequestering particles may have a particle size of 1-5 μm, which may promote deeper airway deposition. The particle size of the sequestering particles may be modulated to target the location of the pathogenic infection, for example, virus infection, in the airways. To increase mucosal diffusion, a micro deployable system may be used for tuned aerodynamic delivery with peptide-functionalized nanoparticles that are released to bind a pathogen, for example, virus.

The surface of the sequestering particles may have been modified to improve sequestering a pathogen, for example, viral particles. The surface modification may be PEGylation, zwitterions, or addition of biomarkers such as CD47.

The surface charge of the sequestering particles may be adjusted to be slightly negative for promoting mucosal transport. Diffusion can be further enhanced by modulating magnitude of surface charge, or incorporating other surface modifications (PEGlyation, zwitterions). The sequestering particles may have a zeta potential less than zero.

The sequestering particle may also be surface functionalized to modulate peptide density. The composition may comprise the protein binding agent at 1-80, 1-70, 1-60, 1-50, 1-40, 1-30, 1-20, 5-80, 5-70, 5-60, 5-50, 5-40, 5-30, 5-20, 10-80, 10-70, 10-60, 10-50, 10-40, 10-30, 10-20, 15-80, 15-70, 15-60, 15-50, 15-40, 15-30, 15-20, 20-80, 20-70, 20-60, 20-50, 20-40 or 20-30 wt %, based on the total weight of the composition.

The sequestering particles may be desiccated. The composition may be a mixture a hydrogel and a dry powder for swellable delivery.

The pathogen-particle aggregates, for example, virus-particle aggregates, may be cleared from the subject by coughing, mucociliary transport, or phagocytes. Aggregates of different sizes may be cleared via different mechanisms. Larger particles, for example, macroparticles, may bind multiple pathogens, for example, viruses, while nanoparticles may bind fewer pathogens, for example, viruses.

In one embodiment, the target virus may be severe acute respiratory syndrome coronavirus 2 (SARS-CoV-2), the capsid protein may be the spike (S) protein and bind a S protein binding domain in angiotensin converting enzyme 2 protein (ACE 2), and the protein binding agent may be a peptide having an amino acid sequence at least 70, 75, 80, 85, 90, 95, 99 or 100% identical to that of the S protein binding domain and blocks the binding of the S protein to the ACE2 protein. The peptide may consist of the amino acid sequence of C-(PEG)₄-IEEQAKTFLDKFNHEAEDLFYQS (SEQ ID NO: 1) or any similar derivative of the hACE2 receptor binding site, C-SVTCKSGDFSCGGRVNRCIPQFWRCDGQVDCDNGSDEQGCP (SEQ ID NO: 2) or any derivative of the CR2 domain of LDL-R, or C-PKTCSQDEFRCHDGKCISRQFVCDSDRDCLDGSDEASCP (SEQ ID NO: 3) or any derivative of the CR3 domain of LDL-R. Upper airway delivery for SARS-COV2 is preferred; upper airway delivery also allows particle clearance via mucocilliary escalator.

The composition may be formulated for different delivery. For example, the composition may be formulated for aerosolized or nebulization delivery. The composition may be formulated for oral and/or nasal administration. The composition may be formulated for delivering the sequestering particles to the upper respiratory tract of the subject. The upper respiratory tract includes nasal cavity, pharynx, larynx, trachea, main stem bronchi, bronchioles and terminal bronchioles. The composition may be formulated for delivering the sequestering particles to the lower respiratory tract of the subject. The lower respiratory tract includes respiratory bronchioles, alveolar ducts, and alveolar sacs/alveoli.

The subject may be an animal, a mammal, or a human. The subject may not have been infected by the target pathogen (e.g., virus). The subject may have been exposed by the target pathogen (e.g., virus). The composition may be administered to the subject prophylactically. The subject may have been vaccinated against the target pathogen (e.g., virus).

For each composition of the present invention, a method for sequestering a target pathogen, for example, viral particles of a target virus, in the respiratory tract of a subject is provided. The method comprises delivering the composition to the respiratory tract of the subject, and forming aggregates of the sequestering particles and the pathogen, for example, viral particles, in the respiratory tract. As a result, the pathogen, for example, viral particles, is sequestered by the sequestering particles.

According the method of the present invention, the target pathogen, for example, virus, may be a respiratory pathogen, for example, respiratory virus. Examples of respiratory viruses include severe acute respiratory syndrome coronavirus 2 (SARS-CoV-2), influenza virus, respiratory syncytial virus (RSV), hantavirus, rhinovirus, parainfluenza virus (PIV), and human metapneumonia virus (hMPV). Examples of respiratory bacteria include Streptococcus pneumoniae, Haemophilus species, Staphylococcus aureus, Nontuberculous mycobacteria, and Mycobacterium tuberculosis. Examples of respiratory fungi include Aspergillus, Cryptococcus, and Pneumocystis.

According the method of the present invention, the protein binding agent may be selected from the group consisting of peptides, aptamers, antibodies, nanobodies, and a combination thereof. The peptide may have about 10-20 amino acids. The peptide may have an amino acid sequence at least 70, 75, 80, 85, 90, 95, 99 or 100% identical to that of a binding domain on a cellular receptor specific for a pathogenic surface protein, for example, capsid protein of a viral particle. The aptamer may have 20 to 60 nucleotides.

According the method of the present invention, the sequestering particles may be selected from the group consisting of polymeric particles, liposomes, lipid/membrane-wrapped particles, alginate, polysaccharide based platforms, and combinations thereof. The polymeric particles may comprise poly(ethylene glycol) diacrylate (PEGDA), poly(acrylic acid), poly lactic-co-glycolic acid, 2-carboxyethyl acrylate or 2-amino ethyl methacrylate. The liposomes are spherical droplets stabilized by a phospholipid membrane. The lipid/membrane-wrapped particles are synthetic particles such as gold, silver, silica, or PEGDA coated with cellular membranes harvested from mammalian cells. The polysaccharide based platforms are particles made from natural hydrogel materials such as starch, alginate, and agarose. The sequestering particles may be swellable biocompatible hydrogel, such as PEGDA, alginate, agarose, hyaluronic acid, gelatin, poly(acrylic acid). The sequestering particles may further comprise a dye or fluorescent molecule. The sequestering particles may have a particle size of 1-10 μm. The sequestering particles may have a particle size of 5-10 μm, which may be suitable for upper airway delivery. The sequestering particles may have a particle size of 1-5 μm, which may promote deeper airway deposition.

According the method of the present invention, the surface of the sequestering particles may have been modified to improve sequestering a pathogen, for example, viral particles. The surface modification may be PEGylation, zwitterions, or addition of biomarkers such as CD47. The surface charge of the sequestering particles may be adjusted to be slightly negative for promoting mucosal transport. Diffusion can be further enhanced by modulating magnitude of surface charge, or incorporating other surface modifications (e.g., PEGylation, zwitterions). The sequestering particles may have a zeta potential less than zero. The sequestering particle may also be surface functionalized to modulate peptide density. The composition may comprise the protein binding agent at 1-80, 1-70, 1-60, 1-50, 1-40, 1-30, 1-20, 5-80, 5-70, 5-60, 5-50, 5-40, 5-30, 5-20, 10-80, 10-70, 10-60, 10-50, 10-40, 10-30, 10-20, 15-80, 15-70, 15-60, 15-50, 15-40, 15-30, 15-20, 20-80, 20-70, 20-60, 20-50, 20-40 or 20-30 wt %, based on the total weight of the composition. The sequestering particles may be desiccated. The composition may be a mixture a hydrogel and a dry powder for swellable delivery.

The method may further comprise removing the pathogen-particle aggregates, for example, virus-particle aggregates, from the subject. The aggregates may be cleared from the subject by coughing, mucociliary transport, or phagocytes. Aggregates of different sizes may be cleared via different mechanisms. Larger particles, for example, microparticles, may bind multiple pathogens (e.g., viruses) while nanoparticles may bind fewer pathogens (e.g., viruses).

The method may further comprise reducing at least 20%, 30%, 40%, 50%, 60%, 70%, 80%, 90%, 95%, 99% or 100% of the target pathogen, for example, viral particles, in the respiratory tract of the subject. In one embodiment, at least 50% of the pathogens, for example, viral particles, in the respiratory tract of the subject are reduced.

According to the method of the present invention, the composition may be formulated for aerosolized or nebulization delivery. The composition may be formulated for oral and/or nasal administration. The composition may be formulated for delivering the sequestering particles to the upper respiratory tract of the subject. The upper respiratory tract includes nasal cavity, pharynx, larynx, trachea, main stem bronchi, bronchioles and terminal bronchioles. The composition may be formulated for delivering the sequestering particles to the lower respiratory tract of the subject. The lower respiratory tract includes respiratory bronchioles, alveolar ducts, and alveolar sacs/alveoli.

According to the method of the present invention, the subject may be an animal, a mammal, or a human. The subject may not have been infected by the target pathogen (e.g., virus). The subject may have been exposed by the target pathogen (e.g., virus). The composition may be administered to the subject prophylactically. The subject may have been vaccinated against the target pathogen (e.g., virus).

### Example 1. Particle Fabrication & Functionalization

Sequestering particles have been prepared by modifying microparticles particles such as PEGDA MPs by attaching a peptide to the macroparticles.

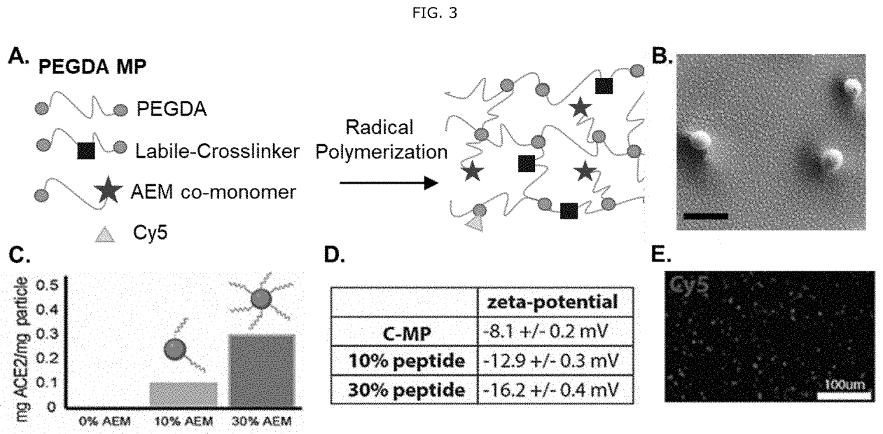

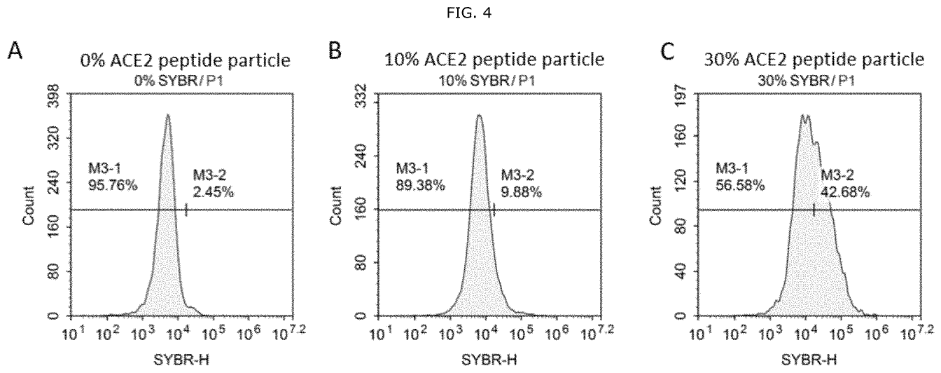

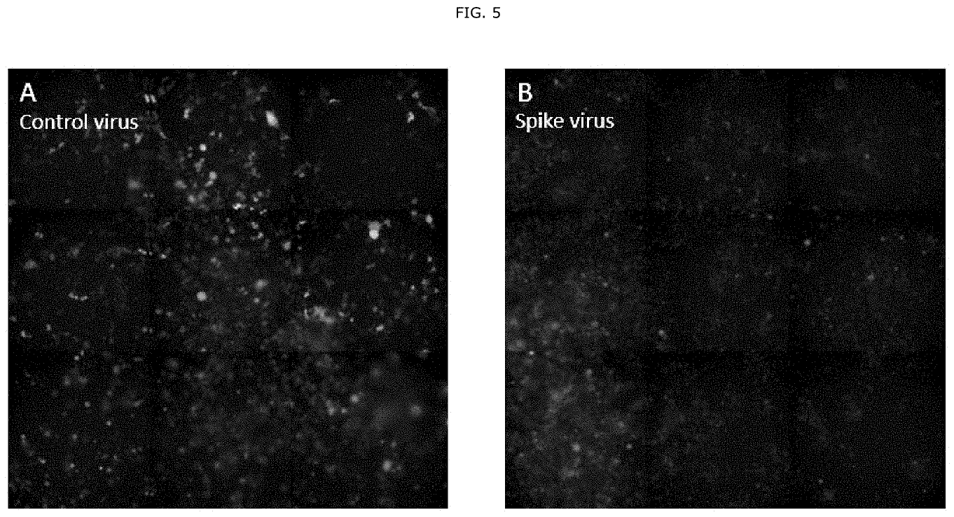

PEGDA MPs have been synthesized using a pre-particle solution containing functional monomers, photoinitiator, and fluorescent dye is emulsified in silicone oil and exposed to UV light to initiate polymerization (FIG. 3A). The pre-particle solution consists of up to 88% wt/wt crosslinker [PEGDA, Mn=700], 1% dye [cyanine 5 (cy5)], 10% functional monomer [2-aminoethyl methacrylate (AEM)], and 1% photoinitiator [diphenyl(2,4,6-trimethylbenzoyl)phosphine oxide (TPO)]. After polymerization, silicone oil is be removed via centrifugation and washing steps in hexane and ethanol. PEGDA hydrogels are highly modular, with co-monomers providing functional groups and degradation control. We have demonstrated that control of the weight % solids (largely PEGDA) can tune modulus, particle elasticity, and swellability.

Alternative acrylate comonomers alter the final conjugation chemistry to link the peptide to ensure options for covalent attachment and others tested include 2-carboxyethyl acrylate (CEA) to provide pendant carboxylates.

Dyes and fluorescent molecules have been used for tracking. Any fluorescent molecule w/thiol or acrylate can be polymerized into the formulation.

In concept, other biocompatible chemistries that a) swell in aqueous conditions, b) allow for further surface functionalization with the peptide and c) are naturally degraded/cleared from the airspaces/body could be used in place of the PEGDA system. Other options proposed include various polymeric particles, liposomes, lipid/membrane-wrapped particles, alginate, and/or polysaccharide based platforms.

FIG. 3B demonstrates our ability to generate representative PEGDA nanoparticles via Cryo-SEM, scale bar 500 nm.

Pendent amines from AEM-containing MPs are used for post-fabrication modifications through carbodiimide chemistry for ACE2 peptide fragment incorporation. MP modification does not change MP size or shape considerably, therefore, particle deposition patterns should not differ. MPs have been modified with ACE2 peptide fragment (ACE2-MP) and MPs without modification (C-MP) were used as a control.

Different amounts of AEM in particle were used to control amount of peptide loading, using constant peptide reaction conditions; equally acceptable alternative approach has been to use constant AEM in particles and vary the peptide reaction conditions. Either approach yields control over final peptide incorporation.

Peptide range has been demonstrated from 0-30%, which corresponds to −30% by mass of peptide to particle. Tuning the amount of peptide will modulate the binding; however, diminishing returns would be expected as the peptide loading increases much more beyond 50% and will be limited by the amount of functional handles present in the formulation, and any steric hinderance on particle surface to incorporate more peptide.

The thiolated ACE2 peptide used in our preliminary data was (C-(PEG)4-IEEQAKTFLDKFNHEAEDLFYQS) (SEQ ID NO: 1). The modified thiol was used for conjugation chemistry and other bioconjugation strategies are acceptable (I.e., click, carbodiimide). The PEG4 spacer was used to give peptide binding flexibility and extend peptide from the particle surface for increased binding. Alternative PEG lengths could be included for any peptide.

Representative zeta potential measurements shown for functionalized ACE2-MPs.

Mucus is predominantly negatively charged (in general, healthy more negatively charged than in diseased mucus); MPs should be negatively charged to enhance diffusion.

Particles have been characterized for size and concentration using thermogravitational analysis (TGA), dynamic light scattering (DLS), scanning electron microscopy (SEM), endotoxin screening with LAL assay, and peptide loading with BCA Protein Assay.

### Example 2. Particle Binding Virus

Flow cytometry results look at binding of a SYBR-stained virus expressing the SARS-COV2 spike protein, which binds to the ACE2 peptide on the particle. The virus was incubated with microparticles for 4 hours, then the mixture was stained with SYBR and then ran on flow cytometry. Microparticles fluoresce far red due to the inclusion of cy5 during formulation, and the virus should fluoresce green due to the SYBR staining. Events were gated around the microparticles based on their far red fluorescence (P1). Shifted signal to the right (increase in green SYBR signal) and an increase in M3-2 gate % indicates bound particle and virus. This demonstrates that (a) more ace2 peptide leads to more virus binding. It is important to note that even the 10% formulation binds virus; (b) the amount of peptide on surface matters in binding virus and is a design feature to tune binding. From our preliminary data, our assay is not completely optimized; concentrations of virus:particle will matter and evaluation of a range of conditions is on-going. Also, the detection limit on this assay has not been optimized, as the SYBR stain is somewhat non-specific. This means particles are binding more virus than what is shown.

### Example 3. Particle Preventing Cell Infection

Cells (HEK 293T ACE2) were exposed to a control virus which does not have the protein that binds to the MPs+MPs (left) or spike expressing virus+MPs that bind spike virus (right) supernatant for 24 hours in vitro. The virus and MPs were incubated for 3 hours and then quickly centrifuged to pellet the MPs (and any virus bound to MPs) while keeping the free virus in the supernatant. The cells were then dosed with the virus-laden supernatant. Both of the viruses encode mCherry protein and therefore red fluorescence in the cells indicates an infected cell. These results demonstrate (a) incubation w/particles that bind spike virus decreases cellular binding and ultimate infection (right) compared to control virus (left); (b) feasibility of invention in preventing cellular infection.

### Example 4. Diffusion of Particles in Mucus Mimic

Particles have been tested in synthetic mucus mimetics. Mucus mimics were formulated according to an existing protocol by Song et al (ACS Biomaterials Sci & Engineering 2021). This method utilizes porcine gastric mucin (PGM, MUC5AC), bovine submaxillary gland mucin (BSM, MUC5B), and a four-arm PEG thiol crosslinker (PEG-4SH). Initially, 4% w/v solutions of mucin or PEG-4SH in PBS were formulated and mixed for two hours. For the healthy mucus mimic, 75% w/w BSM and 25% w/w PGM were combined as the mucin solution. In comparison, the asthmatic mucus mimic utilized 75% w/w PGM and 25% w/w BSM. Equal amounts of mucin and PEG-4SH solutions were combined to yield a final mucus mimic with 2% w/v mucin and 2% w/v PEG-4SH. Multiple Particle Tracking experiments were performed with the ACE2-MPs and C-MPs to determine differences in diffusivity. Particle diffusivity varied in the synthetic mucins as well as the overall zeta potential of the MP formulation. Neutral to slightly negatively charged MPs diffused more effectively in the synthetic mucus compared to positively charged C-MPs.

Although the invention is illustrated and described herein with reference to specific embodiments, the invention is not intended to be limited to the details shown. Rather, various modifications may be made in the details within the scope and range of equivalents of the claims without departing from the invention.

What is claimed:

1. A composition for sequestering viral particles of a target virus in the respiratory tract of a subject, comprising an effective amount of sequestering particles having a protein binding agent on the surface of the sequestering particles, wherein the protein binding agent binds a capsid protein on the surface of the viral particles, whereby the sequestering particles and the viral particles form aggregates.

2. The composition of claim 1, wherein the sequestering particles are selected from the group consisting of polymeric particles, liposomes, lipid/membrane-wrapped particles, alginate, polysaccharide based platforms, and combinations thereof.

3. The composition of claim 1, wherein the sequestering particles comprise poly(ethylene glycol) diacrylate (PEGDA).

4. The composition of claim 1, wherein the target virus is a respiratory virus selected from the group consisting severe acute respiratory syndrome coronavirus 2 (SARS-CoV-2), influenza virus, respiratory syncytial virus (RSV), hantavirus, rhinovirus, parainfluenza virus (PIV), and human metapneumonia virus (hMPV).

5. The composition of claim 1, wherein the target virus is SARS-CoV-2, the capsid protein is a spike (S) protein and binds a S protein binding domain in angiotensin converting enzyme (ACE) 2 protein, and the protein binding agent is a peptide consisting of an amino acid sequence at least 80% identical to the S protein binding domain and blocks the binding of the S protein to the ACE2 protein.

6. The composition of claim 5, wherein the peptide consists of the amino acid sequence of C-(PEG)₄-IEEQAKTFLDKFNHEAEDLFYQS (SEQ ID NO: 1), C-SVTCKSGDFSCGGRVNRCIPQFWRCDGQVDCDNGSDEQGCP (SEQ ID NO: 2), or C-PKTCSQDEFRCHDGKCISRQFVCDSDRDCLDGSDEASCP (SEQ ID NO: 3).

7. The composition of claim 1, wherein the composition comprises the protein binding agent at 10-50 wt %, based on the total weight of the composition.

8. The composition of claim 1, wherein the sequestering particles have a particle size of 1-10 μm.

9. The composition of claim 1, wherein the sequestering particles have a particle size of 1-5 μm.

10. The composition of claim 1, wherein the sequestering particles have a particle size of 5-10 μm.

11. The composition of claim 1, wherein the sequestering particles are desiccated.

12. The composition of claim 1, wherein the sequestering particles has a zeta potential less than zero.

13. The composition of claim 1, wherein the sequestering particles have a surface modification selected from the group consisting of PEGylation, zwitterions, and CD47.

14. The composition of claim 1, wherein the composition is a mixture of a hydrogel and dry powder.

15. The composition of claim 1, wherein the composition is formulated for aerosolized or nebulization delivery.

16. The composition of claim 1, wherein the composition is formulated for oral and/or nasal administration.

17. The composition of claim 1, wherein the subject has been vaccinated against the target virus.

18. A method for sequestering viral particles of a target virus in the respiratory tract of a subject, comprising:
(a) delivering the composition of claim 1 to the respiratory tract of the subject; and
(b) forming aggregates of the sequestering particles and the viral particles in the respiratory tract, whereby the viral particles are sequestered by the sequestering particles.

19. The method of claim 18, further comprising removing the aggregates from the subject.

20. The method of claim 18, further comprising reducing at least 50% of the viral particles in the respiratory tract of the subject.
